(19) United States
(12) Patent Application Publication
(10) Pub. No.: US 2008/0072338 A1
(43) Pub. Date: Mar. 20, 2008

(54) Animal for Drug Efficacy Evaluation, Method for Developing Chronic Obstructive Pulmonary Disease in Animal for Drug Efficacy Evaluation, and Method for Evaluating Drug Efficacy Using the Animal

(75) Inventors: Nobuaki Mizutani, Kyoto-shi (JP); MItsuteru Ishiwara, Sakura-shi (JP)

Correspondence Address: DARBY & DARBY P.C., P.O. BOX 770, Church Street Station, New York, NY, 10008-0770, US

(73) Assignee: Fuji Biomedix Co., Ltd, Tokyo (JP)

(21) Appl. No.: 11/576,137

(22) PCT Filed: Sep. 26, 2005
(86) PCT No.: PCT/JP05/17619

§ 371 Date: Mar. 27, 2007

(30) Foreign Application Priority Data
Sep. 28, 2004 (JP) 2004-281461

(51) Int. Cl. A01K 67/00

(52) U.S. Cl. 800/9

(57) ABSTRACT

The present invention provides an animal for drug efficacy evaluation produced by administering an aqueous solution, in which cigarette smoke is dissolved in water or physiological saline, directly to a lower airway of an animal except a human so as to develop chronic obstructive pulmonary disease; a method for developing chronic obstructive pulmonary disease in an animal for drug efficacy evaluation including administering an aqueous solution, in which cigarette smoke is dissolved in water or physiological saline, directly to a lower airway of an animal except a human; and a method for evaluating drug efficacy including administering a drug to the animal for drug efficacy evaluation and then evaluating the effect of the drug.

### TECHNICAL FIELD

The present invention relates to an animal for drug efficacy evaluation in which there is no inflammation at a nasal cavity and pharynges and chronic obstructive pulmonary disease is developed, a method for developing chronic obstructive pulmonary disease in the animal for drug efficacy evaluation, a method for evaluating drug efficacy of a drug using the animal for drug efficacy evaluation.

### BACKGROUND ART

A conventional method for evaluating drug efficacy of an anti-chronic obstructive pulmonary disease material includes: letting an experimental animal inhale cigarette smoke as chronic obstructive pulmonary disease-developing factor; producing an animal for drug efficacy evaluation in which chronic obstructive pulmonary disease is experimentally developed; and evaluating the drug efficacy (for example, see Patent Document 1). An animal for drug efficacy evaluation disclosed in his document is produced by letting a 6-week-old mouse inhale mainstream cigarette smoke from Kentucky Reference Cigarette 1R1 for 1 to 4 hours a day, 5 days a week, for a total of 6 months, and therefore a long time is required from the start of an experiment to drug efficacy evaluation, so there is a problem in that drug efficacy evaluation cannot be performed efficiently.

Also, in the cigarette smoke-inhalating method, cigarette smoke passes through an oral cavity or a nasal cavity, so there is a possibility to simultaneously develop an inflammation reaction at the parts other than chronic obstructive pulmonary disease-affected part, and it was difficult to produce an animal for drug efficacy evaluation in which an affected part is selectively specified.
[Patent Document 1] Japanese Unexamined Patent Application, First Publication No. 2004-105173

### DISCLOSURE OF INVENTION

Objects of the present invention are to provide an animal for drug efficacy evaluation in which there is no inflammation at a nasal cavity and pharynges and only chronic obstructive pulmonary disease is developed, and a method for developing only chronic obstructive pulmonary disease in the animal for drug efficacy evaluation. Also, an object of the present invention is to provide a method for evaluating selective drug efficacy against chronic obstructive pulmonary disease by administering to a drug to the animal for drug efficacy evaluation.

The present invention provides an animal for drug efficacy evaluation produced by administering an aqueous solution, in which cigarette smoke is dissolved in water or physiological saline, directly to a lower airway of an animal except a human so as to develop chronic obstructive pulmonary disease.

Also, the present invention provides a method for developing chronic obstructive pulmonary disease in an animal for drug efficacy evaluation including administering an aqueous solution, in which cigarette smoke is dissolved in water or physiological saline, directly to a lower airway of an animal except a human.

Also, the present invention provides a method for evaluating drug efficacy including administering a drug to the animal for drug efficacy evaluation and then evaluating the mechanism and the effect of the drug.

According to the present invention, it is possible to reliably provide an animal for drug efficacy evaluation in which only chronic obstructive pulmonary disease is selectively developed, and to rapidly provide an animal for drug efficacy evaluation in order to perform an evaluation method which is useful for the development of drugs and medicines, especially screening of an anti-chronic obstructive pulmonary disease material.

Hereinafter, the advantageous effects of the present invention are described in detail.
(1) In a conventional experimental animal model, it takes more than 24 weeks to develop the symptoms of chronic obstructive pulmonary disease. In contrast, the present invention can produce an animal for drug efficacy evaluation within as a short period as 4 weeks.
(2) In a conventional experimental animal model, cigarette smoke has been inhaled through a nasal cavity. In contrast, in the present invention, an aqueous solution, in which cigarette smoke is dissolved in water or physiological saline, is produced and then administered directly to a lower airway, thereby making it possible to change the concentrations of the components of cigarette smoke in a solution. Accordingly, it is possible to adjust the degree of a clinical state and to produce an animal for drug efficacy evaluation with consistent characteristic.
(3) In a conventional cigarette smoke-inhalating method, cigarette smoke passes through an oral cavity or a nasal cavity, so an inflammation reaction is simultaneously developed at the parts other than chronic obstructive pulmonary disease-affected part. In contrast, in the present invention, a solution in which cigarette smoke is dissolved is administered directly to a lower airway without passing through an oral cavity and a nasal cavity, so it is possible to selectively specify an affected part and to develop only chronic obstructive pulmonary disease.
(4) In the past, an apparatus configured to generate cigarette smoke and an exposure chamber configured to let an animal inhale the smoke have been required for the model production. In contrast, the present invention does not require such large apparatuses, and it is possible to produce an animal for drug efficacy evaluation inexpensively and reliably.

### BRIEF DESCRIPTION OF THE DRAWINGS

FIG. 1 is a schematic diagram showing a method for producing a cigarette smoke solution used in the present invention.

FIG. 2 is an external view of the lungs of a guinea-pig, in which only physiological saline has been administered and nothing is developed, as a comparing object in Example 1.

FIG. 3 is an external view of the lungs of a suffering guinea-pig in Example 1.

FIG. 4 is a micrograph of the pulmonary tissue of the lungs of a guinea-pig, in which only physiological saline has been administered and nothing is developed, as a comparing object in Example 1.

FIG. 5 is a micrograph of the pulmonary tissue of the lungs of a suffering guinea-pig in Example 1.

The reference numerals shown in these figures are defined as follows:

1 represents a cigarette;

2 represents water or physiological saline;

3 represents a reservoir;

4 represents an introducing tube;

5 represents a valve;

6 represents a suctioning pump; and

7 represents a suctioning tube.

### BEST MODE FOR CARRYING OUT THE INVENTION

In recent years, the increase of fatalities from chronic obstructive pulmonary disease, in which cigarette smoking is thought to be a main reason, has become an issue in Europe, the United States, and Japan. To solve this issue, the development of an effective therapeutic method and a therapeutic drug has become a very important medical objective. Therefore, the development of a drug having a therapeutic effect has been desired, and an animal for drug efficacy evaluation for a screening test of a drug has been waited.

In the present invention, it is possible to reliably provide an animal for drug efficacy evaluation in which chronic obstructive pulmonary disease is developed (hereinafter referred to as a “chronic obstructive pulmonary disease-model animal”), which has been desired in the market.

Hereinafter, chronic obstructive pulmonary disease in the present invention will be briefly described.

Chronic obstructive pulmonary disease is among pulmonary diseases including chronic bronchitis and lung emphysema. While some kinds are reversible, chronic obstructive pulmonary disease is generally characterized by a progressive, irreversible airway obstruction, and is accompanied by airway hyperreactivity in many cases. Chronic bronchitis is characterized by chronic wet cough which lasts for three months or more in each year of a consecutive two years. Also, lung emphysema is accompanied by a destructive change of the pulmonary alveoli, and is the abnormal permanent enlargement of the air space which is far away from the terminal bronchioli without obvious fibrosis. The destruction is defined as an irregular enlargement of the respiration-related air space, and regular appearances of the pulmonary acini and the components thereof may be lost.

As described above, chronic obstructive pulmonary disease is characterized by an irreversible airway obstruction, and has a different disease conception from asthma which is a reversible airway obstruction-related disease. As for a drug treatment for bronchial asthma, the inhalation of a steroid is recommended as a first-line drug, and the excellent usefulness thereof is confirmed in Guideline for the Diagnosis and Management of Asthma (NHLBI, 2002), which is a global guideline for the medical treatment of asthma. In contrast, as for a drug treatment for chronic obstructive pulmonary disease, the effect of a steroid is limited, and the use thereof is not generally recommended in Global Initiative for Chronic Obstructive Lung Disease (GOLD; NHLBI/WHO, 1998), which is also the same global guideline as above. In this way, bronchial asthma and chronic obstructive pulmonary disease have different reactivity to a drug.

The risk factors of chronic obstructive pulmonary disease are harmful fine particles due to cigarette smoking and atmospheric pollution, and the reason for developing this disease is thought to be because the exposure to these harmful fine particles maintains a chronic inflammation state in the peripheral airway and the pulmonary alveoli. The aforementioned Global Initiative for Chronic Obstructive Lung Disease (GOLD; NHLBI/WHO, 1998 year) clearly teaches that neutrophil-related inflammation appears in the lungs in chronic obstructive pulmonary disease and that inflammation due to a disproportion between a protease and a protease inhibitor, and oxidation stress are important as the causing and progressing factors.

In the present invention, it is made possible to evaluate the drug efficacy of a drug accurately and over time by selectively providing an animal for drug efficacy evaluation in which only chronic obstructive pulmonary disease is developed, the animal being different from a conventional chronic obstructive pulmonary disease animal model.

A chronic obstructive pulmonary disease animal model of the present invention is produced by administering an aqueous solution, in which cigarette smoke is dissolved in water or physiological saline (hereinafter referred to as a 37 cigarette smoke solution”), directly to a lower airway of an animal except a human; simultaneously administering a cigarette smoke solution and an aqueous solution containing an endotoxin (hereinafter simply referred to as an “endotoxin-containing aqueous solution”) directly to a lower airway of an animal except a human; or administering an aqueous solution, in which cigarette smoke and an endotoxin are dissolved in water or physiological saline (hereinafter referred to as a “cigarette smoke/endotoxin-containing aqueous solution”), directly to a lower airway of an animal except a human. The term “lower airway” here is defined as the trachea, bronchi, bronchioli, alveoli, and so on among the airway. Also, he term “directly administering” is defined as administering a cigarette smoke solution, an endotoxin-containing aqueous solution, or a cigarette smoke/endotoxin-containing aqueous solution directly to a lower airway without touching a nasal cavity.

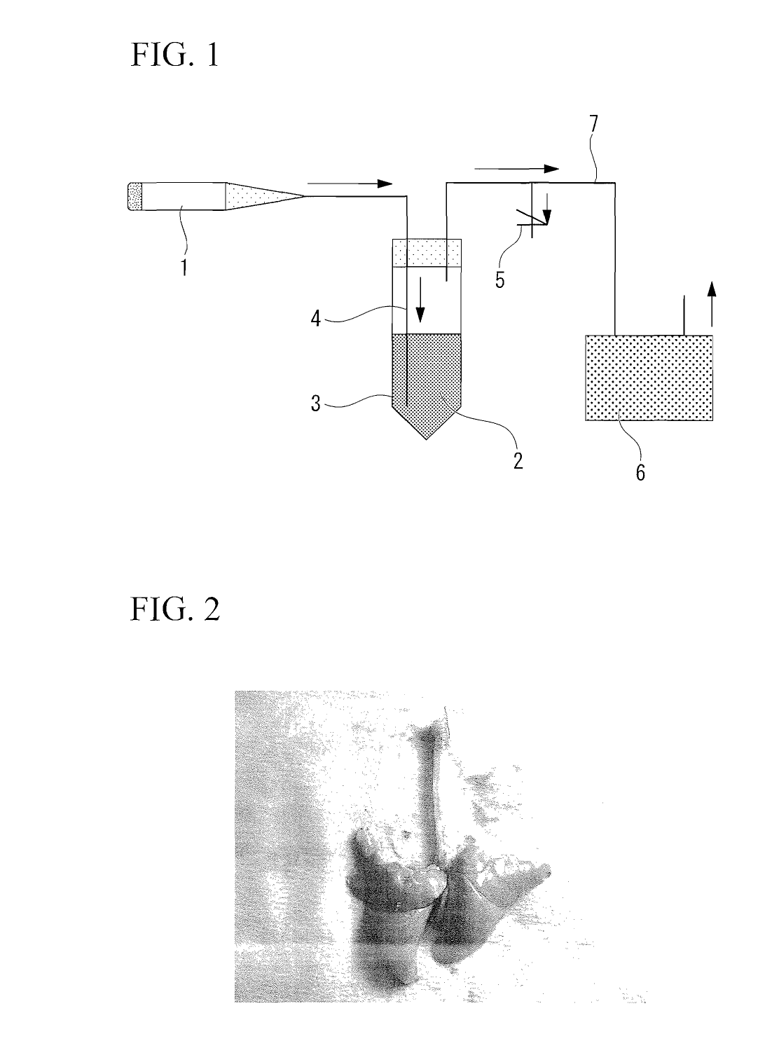

The aforementioned cigarette smoke solution can be produced by the method illustrated in the schematic diagram of FIG. 1, for example. That is, a filter mouthpiece of a cigarette 1 is connected with one end of an introduction tube 4, and the other end of the introduction tube 4 is immersed in water or physiological saline 2 in a reservoir 3. Meanwhile, a suction pump 6 and a suction tube 7 are connected with each other, and the reservoir 3 and the other end of the suction tube 7 are connected with each other without touching the aqueous surface. Herein, a valve 5 is provided on the suction tube 7. After having lighted the cigarette 1, the suction pump 6 is operated, and cigarette smoke is bubbled in water while appropriately opening or closing the valve 5, thereby producing the cigarette smoke solution.

The concentration of cigarette smoke in a cigarette smoke solution and a cigarette/endotoxin-containing aqueous solution is not limited as long as it is sufficient to develop chronic obstructive pulmonary disease in a used animal. For example, chronic obstructive pulmonary disease can be developed by the aspiration of cigarette smoke of one cigarette or more and preferably five cigarettes or more per 10 mL of a solution. Also, the amount of a cigarette smoke solution used is not limited as long as it is sufficient to develop chronic obstructive pulmonary disease in a used animal, and an example thereof is preferably within a range of 20 μL or more and more preferably within a range of 50 μL to 5 mL at one administration per one animal. Water, physiological saline, and a water-soluble organic solvent which does not have a harmful effect on an animal can be included in the aforementioned aqueous solution. Herein, examples of the water-soluble organic solvent include ethanol and dimethyl sulfoxide. The content thereof in the aqueous solution varies depending on a kind of animal used while 0.01 to 10 weight % can be exemplified as a guide.

Next, an endotoxin which is produced by a Gram-negative bacterium used in the present invention will be described.

While cigarettes are thought to be the main reason for the development of chronic obstructive pulmonary disease, a secondary factor such as infection is thought to deteriorate symptoms. Therefore, an object of the simultaneous use of an endotoxin produced by a Gram-negative bacterium is to produce an animal for drug efficacy evaluation which has more similar symptoms to those in real life by adding the secondary factor. The term “an endotoxin produced by a Gram-negative bacterium” here is the endotoxin released from a cell wall of a Gram-negative bacterium, and examples thereof include a lipopolysaccharide. It is preferable that the endotoxin be administered in a solution state, for example while being dissolved in water or physiological saline.

The concentration of an endotoxin produced by a Gram-negative bacterium is not limited as long as it is sufficient to develop chronic obstructive pulmonary disease in a used animal. For example, in water or physiological saline, or a cigarette solution, the concentration is preferably within a range of 10 μg/mL or more and more preferably within a range of 50 μg/mL to 2 g/mL. The endotoxin produced by a Gram-negative bacterium may not be included in water or physiological saline, or a cigarette solution, but is preferably included because of the aforementioned reasons.

As for the number of administrations and the administration period of a cigarette smoke solution, an endotoxin-containing aqueous solution, or a cigarette smoke/endotoxin-containing aqueous solution, optimum conditions vary depending on a kind of experimental animal used. Therefore, it is preferable that a preliminary test be performed to set the conditions.

Also, the number of administrations and the administration period of a cigarette smoke solution, an endotoxin-containing aqueous solution, or a cigarette smoke/endotoxin-containing aqueous solution can be decided by the development degree of chronic obstructive pulmonary disease. The development degree is evaluated by a peak expiratory flow (mL/sec), and the number of administrations and the administration period, in which a peak expiratory flow is reduced by 10% or more in comparison with the physiological saline-administered group, is set. Herein, when the administration of the cigarette solution and the administration of the endotoxin-containing aqueous solution are simultaneously performed, it is preferable that the administration of the endotoxin-containing aqueous solution be performed between the administrations of the cigarette smoke solution.

By performing the administration of an endotoxin produced by a Gram-negative bacterium in addition to the administration of a cigarette smoke solution, it becomes possible to develop chronic obstructive pulmonary disease reliably and in a short period.

The chronic obstructive pulmonary disease-model animal produced in this way has a characterizing feature in that inflammation can be developed at a chronic obstructive pulmonary disease-affected part. For example, when an Evans' Blue solution was administered to the lower airway, which is a characterizing feature of the present invention to confirm a colored part of a living body, the colored part was not confirmed at a nasal part and a nasal cavity but was limited to an oral part and the lower airway, and the ratio thereof was 100%.

Subsequently, a method for administering a cigarette smoke solution, a cigarette smoke solution and an endotoxin-containing aqueous solution, or a cigarette smoke/endotoxin-containing aqueous solution directly to the lower airway of an animal except a human will be described.

In administration, a metal feeding tube can be used. For example, a generally used stainless-steel feeding tube can be used. Also, a metal feeding tube having a preferable size and shape can be selected depending on a kind of animal actually used. It is usually preferable to process the end of a tube so as to reliably perform the direct administration to lower airway. Examples of the processing method include a method to make the end of a stainless-steel feeding tube curved to be easily inserted to lower airway directly. The degree and the shape of a curve vary depending on a kind and a shape of a used animal while it is preferable that a curve be made such that the corresponding part to the curved end and the corresponding part to the base of a feeding tube make an angle of 90° to 150°. In the direct administration, a curved feeding tube is inserted through an oral cavity, one end of the feeding tube is allocated on the upper portion of the lower airway of pharynx of the animal, and the aqueous solution is administered from the other end of the feeding tube directly to the lower airway by the spontaneous respiration of the animal.

As an experimental animal used in the present invention, an animal which has a lower airway and performs pulmonary respiration can be used, and examples thereof include Rodentia such as a guinea-pig, a mouse, a rat, or a hamster; Lagomorpha such as a rabbit; Artiodactyla such as a goat, a sheep, or a pig; Perissodactyla such as a horse or a donkey; Carnivora such as a dog or a cat; and Primates such as a monkey or a chimpanzee. A guinea-pig, a mouse, a rat, a hamster, a rabbit, a monkey, a chimpanzee, a dog, a goat, a sheep, or a pig, which are conventional experimental animals, are preferable, and Rodentia such as a guinea-pig, a mouse, a rat, or a hamster are particularly preferable in consideration of advantages in experimental operations due to the shape and cost of an experimental animal.

In a method for evaluating drug efficacy, a drug is administered to the aforementioned animal for drug efficacy evaluation, and then the mechanism and the effect of the drug are evaluated. The evaluation of the mechanism and the effect may be performed by using a change of respiratory function. The evaluation of chronic obstructive pulmonary disease in a chronic obstructive pulmonary disease-model animal of the present invention is performed by using the change of respiratory function, which is usually used as an evaluation parameter of the disease, i.e. a specific airway resistance, a peak expiratory flow, a tidal volume, a respiratory minute rate, or a respiratory minute volume. These parameters can be measured by known methods. For example, a specific airway resistance can be measured by using a general respiratory function measurement system (for example, PULMOS-I; M.I.P.S) through a flow sensor in accordance with a method described in Journal of Applied Physiology, 1979, Vol. 46, 2, p. 399-406 written by Pennock B E, et al. (hereinafter, referred to as the “method of Pennock B E, et al.”). Also, the measurements of a peak expiratory flow, a tidal volume, a respiratory minute rate, and a respiratory minute volume can be performed by using PULMOS-I; M.I.P.S.

Also, it is a characterizing feature in chronic obstructive pulmonary disease that neutrophils among inflammatory cells permeate a cell membrane, so a inflammatory degree can be confirmed by measuring the cell count of neutrophils.

### EXAMPLES

Hereinafter, Examples will be described to explain the present invention in more detail, but the present invention is not limited thereto.

### Example 1

<Production of Chronic Obstructive Pulmonary Disease-Model Animal>

In the case where an experimental animal is a guinea-pig, the production of a chronic obstructive pulmonary disease-model animal and the evaluation results of a drug with this animal are described.

1. Preparation of Cigarette Smoke Solution

The cigarette solution was prepared by using the apparatus in the schematic diagram illustrated in FIG. 1.

The desire cigarette smoke solution was prepared by suctioning cigarette smoke into physiological saline with a suctioning time of about 5 minutes per 1 cigarette as a guide by using the following:

1) the number of cigarettes used: 40 cigarettes,

2) the amount of physiological saline: 40 mL

3) the intake volume and the intake time of a pump: a suctioning pump (an intake volume: 6.5 mL/min.).

2. Preparation of Lipopolysaccharide Solution

The solution, in which a lipopolysaccharide is used as an endotoxin produced by a Gram-negative bacterium, was prepared as follows. That is, 50 mg of a lipopolysaccharide was dissolved in physiological saline to prepare a lipopolysaccharide solution having a concentration of 500 μg/mL.

3. Development of Affected Animal

An affected animal was developed by using the cigarette smoke solution and the lipopolysaccharide solution, which were prepared by 1. and 2., as follows.

1) The used animal: 6 Hartley-strain male guinea-pigs (purchased at 4-week-old and preliminarily housed for 1 week before the test)

2) The administration mode and the administration period: The cigarette smoke solution (200 μL/animal/time) was administered once a day and for 4 consecutive days, and the lipopolysaccharide solution (500 μL/animal/day) was administered on day 5. This was referred to as 1 cycle, and the same operation was repeated on day 6 and day 11. Then, the cigarette smoke solution (200 μL/animal/time) was administered on day 16 to day 19.

3) The administration method: In the direct administration, a curved feeding tube was inserted through the oral cavity, one end of the feeding tube was allocated on the upper portion of the lower airway of the pharynx of the animal, and the cigarette smoke solution and the lipopolysaccharide solution were administered from the other end of the feeding tube directly to the lower airway by the spontaneous respiration of the animal.

4. Evaluation of Disease

The evaluation of disease was performed by measuring a specific airway resistance, a peak expiratory flow, a tidal volume, a respiratory minute rate, and a respiratory minute volume on day 20 for the animal developed in 3.

The results are shown in Table 1 (the average value of 6 animals).

TABLE 1
Change of respiratory function (guinea-pig)

| | Specific | Peak | | | Respiratory |
|---|---|---|---|---|---|
| | airway | expiratory | Tidal | Respiratory | minute |
| Administration | resistance | flow | volume | minute rate | volume |
| group | (cmH₂O/sec) | (mL/sec) | (mL) | (breaths/min) | (mL/min) |
| Physiological | 1.0 | 17.5 | 4.4 | 95.5 | 442 |
| saline | | | | | |
| Cigarette solution + | 2.0 | 8.1 | 2.7 | 72.7 | 200 |
| lipopolysaccharide solution | | | | | |

By the present example, the increase of specific airway resistance and the decrease of peak expiratory flow, tidal volume, respiratory minute rate, and respiratory minute volume were found in the suffering animal, and the development of chronic obstructive pulmonary disease was confirmed.

Therefore, the results of Example 1 revealed that, according to the present invention, it is possible to produce an experimental chronic obstructive pulmonary disease-model animal having severe symptoms in a short period without using a large apparatus such as an exposure chamber while inflammation is not caused at a part such as an oral cavity or a nasal cavity.

<Observation of Pulmonary Organ and Pulmonary Tissue>

As for chronic obstructive pulmonary disease of the aforementioned developed animal, photographs of a pulmonary organ and micrographs of a pulmonary tissue in the guinea-pig are shown.

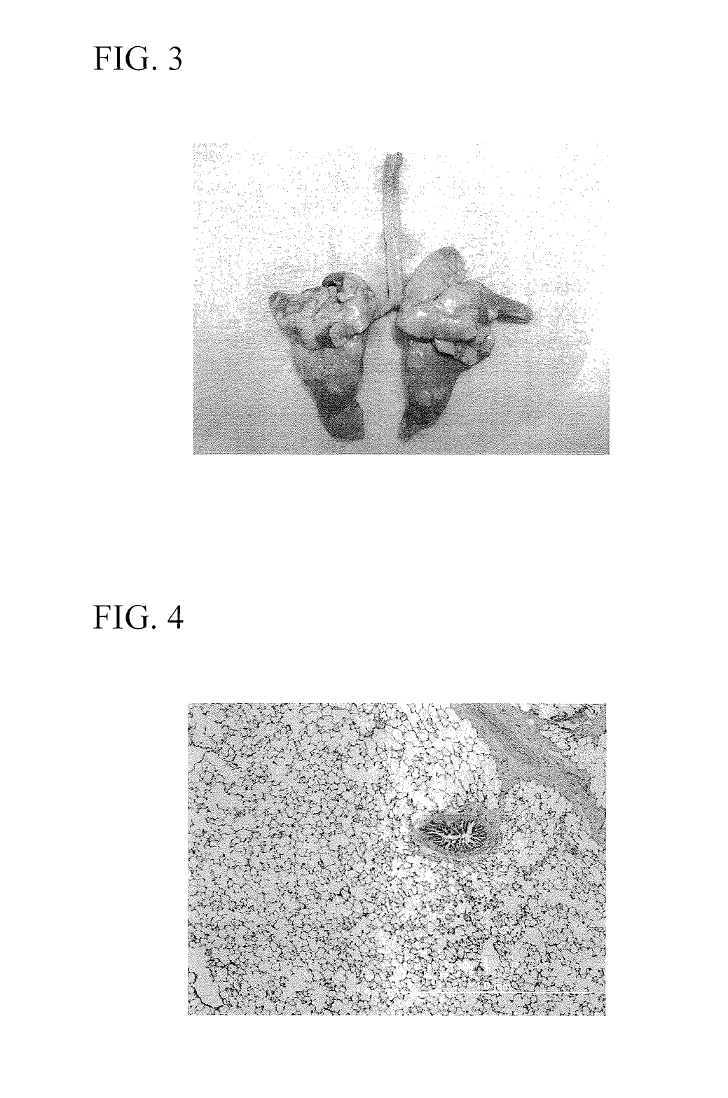

That is, FIG. 2 is an external view of the lungs of a guinea-pig in which only physiological saline has been administered and nothing is developed, and FIG. 3 is an external view of the lungs of a suffering guinea-pig. In the developed guinea-pig of the present example, lung hyperinflation, which is a characterizing feature of chronic obstructive pulmonary disease, was confirmed.

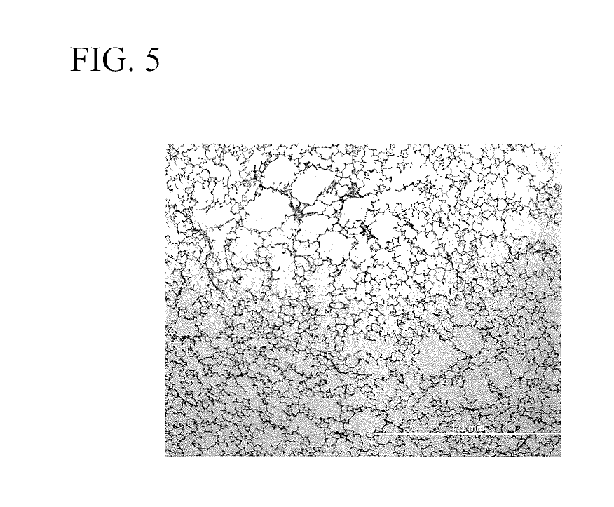

Also, FIG. 4 is a micrograph of the pulmonary tissue of the lungs of a guinea-pig in which only physiological saline has been administered and nothing is developed, and FIG. 5 is a micrograph of the pulmonary tissue of the lungs of a suffering guinea-pig in the present example. Dilation of the alveolar spaces and destruction of the alveolar walls, which are characterizing features of chronic obstructive pulmonary disease, was confirmed in the suffering guinea-pig of the present example in comparison with the normal guinea-pig.

By the photographs of a pulmonary organ and the micrographs of a pulmonary tissue, the symptoms of chronic obstructive pulmonary disease were confirmed, and it was revealed that this disease was developed in the guinea-pigs of the present example.

<Measurement of Number of Neutrophils in Bronchoalveolar Lavage Fluid>

It is known in chronic obstructive pulmonary disease that neutrophils among inflammatory cells permeate a cell membrane so as to develop inflammation. Then, the number of neutrophils was measured for the purpose of confirming the occurrence of chronic obstructive pulmonary disease in the experimental model of the present invention.

5. Collection Method of Leukocyte

The suffering guinea-pig was sacrificed by exsanguination after the measurement of the respiratory function. Then the vessel was perfused by introducing physiological saline through the pulmonary artery, and bronchoalveolar lavage (BAL) was performed by introducing physiological saline (solution volume: 5 mL×2/animal) through an intubated tracheal cannula, and the BAL fluid (BALF) was collected. The BALF was centrifuged with a refrigerated centrifuge (4° C., 400×g, 10 minutes) followed by performing a hemolysis treatment, and was centrifuged again under the same conditions. The obtained pellet was suspended in physiological saline.

6. Measurement of Total Number of Leukocytes

Tulk solution (100 μL) was added to BALF (25 μL) obtained in 5. Then, the total number of leukocytes was counted by using a Burker-Tulk hemocytometer (produced by ERMA Inc.).

7. Production of Cell Smear Sample and Measurement of Number of Neutrophils

On the basis of the total leukocyte number obtained in 6., the leukocyte concentration in BALF was adjusted to be 3×10⁵ cell/mL, and 25 mL was sampled therefrom so as to precipitate the cells on a slide glass. The smear samples were stained with Diff-Quick procedure, and the number of neutrophils was counted microscopically. The results are shown in Table 2 (the average values of 6 animals).

TABLE 2
Number of neutrophils (×10²/μL)

| | Number of |
|---|---|
| Administration group | neutrophils |
| Physiological saline | 1.9 |
| Cigarette solution + lipopolysaccharide solution | 24.0 |

In the present example, it was found that the number of neutrophils was increased in the group in which the cigarette solution and the lipopolysaccharide solution were administered in comparison with the non-stimulated group and that chronic obstructive pulmonary disease was developed.

### Example 2

### Confirmation of Effect of Known Drug Theophylline

For the purpose of revealing the effectiveness of the present invention as a method for evaluating drug efficacy, the effect was confirmed by using theophylline which is known as a therapeutic drug for chronic obstructive pulmonary disease.

In the present example, the preparation of a cigarette smoke solution, the preparation of a lipopolysaccharide solution, the development of suffering animal, and the evaluation of the disease were performed in the same ways as in Example 1. Herein, theophylline (10 mg/kg) was orally administered 1 hour before the administration of the cigarette solution and the LPS solution.

The results are shown in Table 3 (the average values of 6 animals).

TABLE 3
Change of respiratory function due to theophylline administration

| | Specific | Peak | | | Respiratory |
|---|---|---|---|---|---|
| | airway | expiratory | Tidal | Respiratory | minute |
| Administration | resistance | flow | volume | minute rate | volume |
| group | (cmH₂O/sec) | (mL/sec) | (mL) | (breaths/min) | (mL/min) |
| Physiological saline | 1.0 | 16.6 | 4.0 | 109 | 428 |
| Cigarette solution + | 2.4 | 11.2 | 3.0 | 93 | 276 |
| lipopolysaccharide solution | | | | | |
| Theophylline | 1.1 | 19.0 | 3.7 | 124 | 457 |
| (Cigarette solution + | | | | | |
| lipopolysaccharide solution) | | | | | |

By the present example, the improvement effect of respiration function due to theophylline was confirmed in an evaluation method of the present invention, and it was found that a method for evaluating drug efficacy of the present invention is useful as a method for evaluating chronic obstructive pulmonary disease.

### Example 3

### Production of a Chronic Obstructive Pulmonary Disease-Model Animal

In the same ways as Example 1, a chronic obstructive pulmonary disease-model animal was produced by using the experimental animals of a dog, a sheep, and a monkey. As the confirmation of the development, the number of neutrophils was measured in the same way as Example 1.

The results are shown in Table 4 (the average values of 6 animals).

TABLE 4
Numbers of neutrophils in each of experimental animals (/μL)

| Animals | Administration group | Number of neutrophils |
|---|---|---|
| Dog | Physiological saline | 30 |
| | Cigarette solution + | 155 |
| | lipopolysaccharide solution | |
| Sheep | Physiological saline | 10 |
| | Cigarette solution + | 75 |
| | lipopolysaccharide solution | |
| Monkey | Physiological saline | 11 |
| | Cigarette solution + | 60 |
| | lipopolysaccharide solution | |

By the present example, it was found that chronic obstructive pulmonary disease was developed in each of the experimental animals in the group in which the cigarette solution and the lipopolysaccharide solution.

### INDUSTRIAL APPLICABILITY

A chronic obstructive pulmonary disease-model animal of the present invention can be used for the development of drugs and medicines, especially screening of an anti-chronic obstructive pulmonary disease material. Therefore, it is industrially useful.

1-14. (canceled)

15. A method for developing chronic obstructive pulmonary disease in an animal for drug efficacy evaluation, including administering an aqueous solution, in which cigarette smoke is dissolved in water or physiological saline, directly to a lower airway of an animal except a human, wherein
The direct administration includes: inserting a feeding tube through an oral cavity; allocating one end of the feeding tube on the upper part of the lower airway of the pharynx of the anima; and administering the aqueous solution from the other end of the feeding tube directly to the lower airway by the spontaneous respiration of the animal, and
The aqueous solution includes an endotoxin which is produced by a Gram-negative bacterium.

16. (canceled)

17. The method for developing chronic obstructive pulmonary disease in an animal for drug efficacy evaluation according to claim 15, wherein the endotoxin which is produced by the Gram-negative bacterium is a lipopolysaccharide.

18. The method for developing chronic obstructive pulmonary disease in an animal
for drug efficacy evaluation according to claim 15, wherein the aqueous solution includes a water-soluble organic solvent.

19-22. (canceled)

23. A method for producing an animal for drug efficacy evaluation, in which chronic obstructive pulmonary disease is developed, including administering an aqueous solution, in which cigarette smoke is dissolved in water or physiological saline, directly to a lower airway of an animal except a human, wherein
The direct administration includes: inserting a feeding tube through an oral cavity; allocating one end of the feeding tube on the upper part of the lower airway of the pharynx of the animal; and administering the aqueous solution from the other end of the feeding tube directly to the lower airway by the spontaneous respiration of the animal, and
The aqueous solution includes an endotoxin which is produced by a Gram-negative bacterium.

24. The method for producing an animal for drug efficacy evaluation, in which chronic obstructive pulmonary disease is developed, according to claim 23, wherein the endotoxin which is produced by the Gram-negative bacterium is a lipopolysaccharide.

25. The method for producing an animal for drug efficacy evaluation in which chronic obstructive pulmonary disease is developed, according to claim 23, wherein the aqueous solution includes a water-soluble organic solvent.
